(19) United States
(12) Patent Application Publication
(10) Pub. No.: US 2010/0209536 A1
(43) Pub. Date: Aug. 19, 2010

(54) Methods and Kits for Use in the Treatment and Prevention of Diseases or Conditions of the Immune System or Infectious Diseases

(75) Inventors: Scott T. Weiss, Chestnut Hill, MA (US); Augusto A. Litonjua, Jamaica Plain, MA (US); Benjamin Raby, Newton Highlands, MA (US)

Correspondence Address: CLARK & ELBING LLP, 101 FEDERAL STREET, BOSTON, MA, 02110, US

(73) Assignee: The Brigham and Women's Hospital, Inc., Boston, MA (US)

(21) Appl. No.: 12/443,135

(22) PCT Filed: Sep. 24, 2007
(86) PCT No.: PCT/US2007/020579

§ 371 Date: May 6, 2009

Related U.S. Application Data
(60) Provisional application No. 60/847,336, filed on Sep. 26, 2006.

(51) Int. Cl. A61K 31/59; A61K 33/30; C07C 401/00; A61P 37/06; A61P 37/08; A61P 17/00; A61P 17/06; A61P 29/00; A61P 3/10; A61P 31/06; A61P 33/00

(52) U.S. Cl. 424/641; 514/167; 514/168; 552/653

(57) ABSTRACT

This invention provides methods of treating and preventing diseases or conditions of the immune system or infectious disease, involving administration of vitamin D and/or one or more antioxidants (e.g., vitamin E and/or zinc).

### FIELD OF THE INVENTION

This invention relates to methods of treating and preventing diseases or conditions of the immune system or infectious disease, involving administration of vitamin D and/or one or more antioxidants (e.g., vitamin E and/or zinc).

### BACKGROUND OF THE INVENTION

Asthma and allergies are important public health problems in industrialized countries. Asthma, for example, affects over 14 million people and is the most common chronic disease of childhood in the United States. While increases in asthma prevalence over the past decades may have stabilized in some countries, recent data from the United States and other countries suggest that the prevalence continues to rise in some developed countries (Mannino et al., MMWR Surveil. Sum. 51:1-13, 2002; Carter et al., Ann. Allergy Asthma Immunol. 94:634-639, 2005; Braun-Farhlander et al., Eur. Respir. J. 23:407-413, 2004; Ronchetti et al., Eur. Respir. J. 17:881-886, 2001; Verlato et al., J. Allergy Clin. Immunol. 111:1232, 1238, 2003). There are no clear reasons for the increase in prevalence of asthma and allergies in developed countries, but it is likely that a changing environment and behaviors associated with “westernized” lifestyle contribute to the problem. For example, decreased infections in early childhood may predispose to the development of asthma and allergies. Other factors have been implicated in the development of these conditions, including family history and exposure to allergens, which may all increase the risk for asthma and allergies, while exposure to endotoxin, farm animals, and pets may decrease the risk. Changes in diet associated with a western lifestyle may also explain some of these trends.

Vitamin D₃ (cholecalciferol) is a vital nutrient available from food sources (e.g., fortified milk) and nutritional supplements. The skin also can make vitamin D when exposed to sunlight. It has become clear that a large proportion of Americans, especially in the northeastern U.S., have inadequate vitamin D intake, as reflected by serum 25D levels (Hollis, J. Nutr. 135:317-322, 2005). Antioxidants, such as Vitamin E, Vitamin A, and zinc, can be found in numerous food sources (e.g., legumes and vegetables) as well as in nutritional supplements, and the importance of intake of antioxidants such as these to maintaining optimal health has been much emphasized in recent years.

### SUMMARY OF THE INVENTION

The invention provides methods of treating or preventing diseases or conditions of the immune system or infectious disease, which involve (i) administration of at least 400 IU of vitamin D (e.g., 400 IU to 10,000 IU, 1,000 IU to 6,000 IU, or 2,000 IU to 4,000 IU), and/or (ii) administration of one or more antioxidants (e.g., vitamin E (e.g., 200 IU to 4,000 IU, 300 IU to 2,500 IU, 400 IU to 1,000 IU, or 500 IU to 800 IU) and/or zinc). Treatment according to the methods of the invention can be carried out, for example, daily, and optionally can be carried out by administration of the vitamin D and/or one or more antioxidants in a single dosage form. In one example, the vitamin D and/or one or more antioxidants are administered together in the form of a multivitamin. The vitamin D and/or one or more antioxidants can be administered, e.g., orally or by inhalation.

An example of a disease or condition of the immune system that can be treated or prevented, according to the invention, is asthma, which can be treated in adults or children. In another example, the vitamin D and/or one or more antioxidants is administered to a pregnant woman and the treatment or prevention of a disease or condition of the immune system is directed to the child to whom the pregnant woman gives birth. Another example of a patient that can be treated is a child having steroid-resistant asthma. Treatment according to the invention may be used, for example, to improve the response to inhaled steroids in such a patient. In further examples, the disease or condition of the immune system is an autoimmune disease, or is selected from the group consisting of allergic rhinitis, eczema, psoriasis, food allergy, type-1 diabetes mellitus, ulcerative colitis, Crohn's disease, rheumatoid arthritis, multiple sclerosis, systemic lupus erythematosus, bullous pemphigoid, and myasthenia gravis. Examples of infectious diseases that can be treated or prevented, according to the invention, are tuberculosis and parasitic infection.

The invention also includes the use of Vitamin D (optionally in combination with one or more antioxidants, such as those described herein, or other agents described herein) in the prevention or treatment of diseases or conditions such as those described herein (e.g., asthma, as well as the conditions listed in the paragraph immediately above), or in the preparation of medicaments for use in preventing or treating such diseases or conditions.

The invention also provides kits or pharmaceutical containers that include at least 400 IU of vitamin D, for use in a single dosage (e.g., 400 IU to 10,000 IU, 1,000 IU to 6,000 IU, or 2,000 IU to 4,000 IU). Optionally, the kits or pharmaceutical containers can include multiple single dosages of vitamin D. Further, the kits or pharmaceutical containers can also include one or more antioxidants (e.g., vitamin E (e.g., at least 200 IU vitamin E (e.g., 200 IU to 4,000 IU, 300 IU to 2,500 IU, 400 IU to 1,000 IU, or 500 IU to 800 IU), for use in a single dosage) and/or zinc). Optionally, such kits or pharmaceutical containers can include multiple single dosages of vitamin E.

The invention also provides devices (e.g., nebulizers, spray devices, or other inhalers) that include vitamin D for administration by inhalation. These devices can include vitamin D in, e.g., the dosage amounts noted elsewhere herein.

The invention provides several advantages. For example, in providing approaches to treating and preventing asthma, both in patients currently suffering from asthma, as well as prenatally, the invention contributes to the progress in managing what is a very common, chronic disease that is increasing in prevalence.

Other features and advantages of the invention will be apparent from the following detailed description, the drawings, and the claims.

### BRIEF DESCRIPTION OF THE DRAWINGS

FIG. 1 is a graph showing the approximate probability of recurrent wheeze in children at age 3 years, according to their mothers' intake of vitamin D during pregnancy (n=1,194 mother-child pairs). The top band of dots (Yes) represents children who developed recurrent wheeze, while the bottom band (No) represents children who did not. Vertical lines show centiles of maternal vitamin D intake.

FIG. 2 is a graph showing the associations between maternal and child intakes of vitamin D and risk of recurrent wheeze in children at age 3 years (n=1,194 mother-infant pairs). Groups are split at 400 IU vitamin D for mothers (during pregnancy) and 200 IU vitamin D for children (at age 2 years). Odds ratios are adjusted for the 12 factors in multivariate model 2, Table 2.

### DETAILED DESCRIPTION OF THE INVENTION

The invention provides methods and kits for use in the treatment or prevention of diseases or conditions of the immune system (e.g., autoimmune diseases, such as asthma), as well as infectious disease. As described further below, the methods of the invention involve the administration of high dosages of vitamin D (e.g., vitamin D₃ (cholecalciferol); vitamin D₂ (ergocalciferol); calcidiol (25-hydroxycholecalciferol or 25-hydroxy-vitamin D₃); or calcitriol (1,25-dihydroxycholecalciferol or 1,25-dihydroxy-vitamin D₃)) and/or one or more antioxidants (e.g., vitamin E and/or zinc), optionally in combination with each other and/or other agents.

An exemplary disease that can be treated or prevented, according to the invention, is asthma. In one example, asthma is prevented in a child by administration of vitamin D and/or one or more antioxidants (e.g., vitamin E or zinc; optionally in combination with one or more other agents) to the mother of the child during her pregnancy with the child. Vitamin D and/or one or more antioxidants (and optional additional agents) can also be administered to children or adults to treat or prevent asthma in such patients. In one example, the patient is steroid-resistant, and treatment according to the invention increases the efficacy of steroids administered to the patient. Such patients include adults, as well as childhood asthmatics (e.g., children 2-18, 4-16, or 5-12 years old).

In addition to asthma, exemplary diseases or conditions of the immune system that can be treated or prevented, according to the invention, include allergic rhinitis, eczema, psoriasis, food allergy, type-1 diabetes mellitus, ulcerative colitis, Crohn's disease, rheumatoid arthritis, multiple sclerosis, systemic lupus erythematosus, bullous pemphigoid, and myasthenia gravis. Many of these diseases or conditions can also be classified as either TH-1 or TH-2-mediated diseases, as is known in the art. Examples of infectious diseases that can be treated or prevented, according to the invention, include tuberculosis and parasitic diseases.

As is noted above, the methods of the invention involve the administration of high dosages of vitamin D (in any form, such as those listed above) and/or one or more antioxidants, such as vitamin E or zinc. In one example of these methods, at least 400 IU of vitamin D is administered (e.g., 400-10,000, 500-8,000 IU, 1,000-6,000 IU, 2,000-4,000 IU, or 2,500-3,500 IU). In a second example, at least 200 IU of vitamin E is administered (e.g., 200-4,000 IU, 300-2,500 IU, 400-1,500 IU, 500-900 IU, or 600-700 IU). In a third example, at least 400 IU of vitamin D (e.g., 400-10,000, 500-8,000 IU, 1,000-6,000 IU, 2,000-4,000 IU, or 2,500-3,500 IU) and at least 200 IU of vitamin E (e.g., 200-4,000 IU, 300-2,500 IU, 400-1,500 IU, 500-900 IU, or 600-700 IU) are administered. These dosages are typically administered on a daily basis, but administration regimens may be altered as determined to be appropriate by a medical professional.

Antioxidants in addition to vitamin E (tocotrienol or tocopherol) that can be used in the invention include, for example, zinc, copper, vitamins (e.g., vitamin A (retinol) and vitamin C (ascorbic acid)), vitamin co-factors and minerals (e.g., coenzyme Q10 and manganese), hormones (e.g., melatonin), carotenoid terpenoids (e.g., lycopene, lutein, α-carotene, β-carotene, zeaxanthin, astaxanthin, and canthaxantin), non-carotenoid terpenoides (e.g., eugenol), flavonoid polyphenolics (bioflavonoids, e.g., flavonols, flavones, flavanones, flavan-3-ols, isoflavone phytoestrogens, and anthocyanins), and phenolic acids and their esters.

As is noted above, treatment according to the invention can, optionally, be carried out in combination with other, known treatment regimens for a given disease or condition. Thus, in one example, a patient may be a steroid-resistant (SR) asthmatic. In this case, the administration of vitamin D and/or one or more antioxidants (e.g., vitamin E or zinc) can be carried out in conjunction with steroid treatment. Appropriate steroids that can be administered in such combination methods include, for example, methylprednisolone, dexamethasone, beclomethasone, budesonide, and fluticasone. In another example, a patient may be suffering from systemic lupus erythematosus (SLE) and the administration according to the invention can, optionally, be carried out in conjunction with administration of an antimetabolite (e.g., methotrexate). In an additional example, a patient may be suffering from rheumatoid arthritis (RA), systemic lupus erythematosus (SLE), or myasthenia gravis, and the administration according to the invention can, optionally, be accompanied by administration of an immunosuppressant (e.g., hydroxychloroquine, methotrexate, 6-mercaptopurine, azathioprine, cyclosporine, or cyclophosphamide).

Administration of vitamin D and/or one or more antioxidants (e.g., vitamin E and/or zinc; and optionally other agents) can be carried out by separate administration of the agents, at different times; co-administration of the agents at about the same time; or administration of the agents together in a single dosage form. Further, when used in combination, dosages of the agents may or may not be adjusted, as determined to be appropriate by those of skill in the art. The therapeutic agents of the invention can be administered in the form of, e.g., a pill, tablet, or capsule. Other appropriate formulations can be used, as can be determined by those of skill in the art.

In one example, one or more agents administered in carrying out the methods of the invention are in the form of a multivitamin supplement (e.g., in the case of pregnant patients, a prenatal vitamin). In the latter example, the supplement may also include folate (e.g., 40 μg), calcium (e.g., 250 mg), and/or iron (e.g., 30 mg). Additional components of such a supplement may include magnesium (e.g., 320 mg), vitamin C (e.g., 65 mg), vitamin A (e.g., 800 μg or 8,000 IU), and/or B vitamins (e.g., vitamin B6 (2.2 μg, thiamin, and/or riboflavin)), optionally in combination with other vitamins, minerals, or other components of multivitamin supplements.

In another example, administration of vitamin D (and/or vitamin E and/or additional antioxidants) according to the invention is carried out by inhalation. This approach may be particularly advantageous in the treatment of patients with asthma. Formulation of vitamin D for administration by inhalation can be carried out using standard methods (see, e.g., Remington's Pharmaceutical Sciences (18^{th} edition), ed. A. Gennaro, 1990, Mack Publishing Co., Easton, Pa.). As examples, vitamin D can be mixed with an inert lipid and delivered via metered dose inhaler or as a fine powder mist.

More specifically, nebulizers employ drug in droplet form, in solution or suspension, with a pharmaceutically acceptable liquid carrier. Examples of this approach, such as jet nebulization, are described, e.g., in Flament et al., Drug Development and Industrial Pharmacy 21(20):2263-2285, 1995. Briefly, in such methods, air is passed rapidly through a narrow orifice of a tube by the use of a pump, the pressure of the air falls, creating a vacuum, which results in suction of liquid contained in a reservoir connected with the tube. The suctioned liquid is thus reduced to a fine spray or mist that can be inhaled. Vitamin D can be administered, according to the present invention, by use of approaches employing a nebulizer, such as those described above.

In another specific example, vitamin D can be administered by inhalation of dry powdered formulations. Aerosols are dry powder formulations that usually are delivered via pressurized, metered dose inhalers (pMDIs). Aerosol formulation techniques, which can be applied for use in the present invention, are described, e.g., by Sciarra, “Aerosols,” Chapter 92 in Remington's Pharmaceutical Sciences, 16^{th} edition (ed. A. Osol), pp. 1614-1628. Alternatives to pMDIs can also be used, including dry powder inhalers, spacer devices, and holding chambers (see, e.g., Malcolmson et al., PSTT 1(9):394-398, 1998, and Newman et al., “Development of New Inhalers for Aerosol Therapy,” in Proceedings of the Second International Conference on the Pharmaceutical Aerosol, pp. 1-20).

Vitamin D can be administered by inhalation as a sole therapeutic agent or can be mixed with other agents (e.g., a steroid or any other agent used to treat asthma or an agent noted elsewhere herein, e.g., one or more antioxidants, such as those described herein).

The invention also includes kits and pharmaceutical containers (e.g., sterile plastic bottles) that can be used in carrying out the methods described above and elsewhere herein. These kits and containers can include one or more of the therapeutic agents described herein, optionally with instructions to use the agents in methods such as those described herein. The kits and containers can include the agents in separate forms (e.g., a pill, tablet, or capsule) or one or more agents included in the kits and containers of the invention can be combined within a single form (e.g., a pill, tablet, or capsule). The agents can be present in the kits in amounts that readily facilitate carrying out the methods of the invention (see, e.g., the amounts set forth above). The kits or containers of the invention can include agents for use over any convenient period of time, e.g., 1, 2, 3, or 4 weeks, or 1, 2, or 3 months. The invention also includes devices for administration of vitamin D by inhalation (in, e.g., nebulized or aerosolized form), as described above, such as nebulizers and spray devices.

The invention is based, in part, upon the studies described below. The first two show that maternal intake of vitamin D and antioxidants, such as vitamin E or zinc, during pregnancy is associated with lower risks for wheezing illnesses in 2-year old children. The third study shows that low serum Vitamin D levels are associated with increased asthma exacerbations among children using regular inhaled corticosteroids.

### Project Viva: Vitamin D Study

### Design and Participants

Project Viva is a prospective cohort study examining prenatal factors in relation to outcomes of pregnancy and child health. Participants were recruited at eight obstetric offices of Harvard Vanguard Medical Associates, a large multispecialty urban/suburban group practice in eastern Massachusetts. At the 1^{st} study visit, directly after the woman's initial clinical prenatal visit, we obtained informed consent, administered a brief interview, and provided a take-home self-administered questionnaire. At the 2^{nd} study visit, at 26 to 28 weeks gestation, we again administered a brief interview and provided a questionnaire. Project Viva participants gave birth at Brigham and Women's Hospital or Beth Israel Deaconess Medical Center in Boston. Within 3 days after delivery, we interviewed the mother. We reassessed mothers and children at 6 months, 1 year, and annually thereafter. At the time of this analysis, age 3 visits were almost complete.

Exclusion criteria included multiple gestation (e.g., twins), inability to answer questions in English, plans to move out of the area before delivery, and gestational age greater than 22 completed weeks at initial prenatal clinical appointment. Additional details of recruitment and follow-up have been presented elsewhere (Yunginger et al., Am. Rev. Respir. Dis. 146:888-894, 1992). Among 2,128 delivered infants in Project Viva, we excluded 228 because of missing first and second trimester diet assessment data and an additional 37 with a gestation of less than 34 completed weeks. Mothers of 368 of the remaining 1863 participants did not provide informed consent for child follow-up through age 3 years. Of the remaining 1,495 infants, 76 were lost to follow-up and 225 have not yet exited the 3-year time-window, leaving an analysis sample of 1,194 participants. Human subjects committees of Harvard Pilgrim Health Care, Brigham and. Women's Hospital, and Beth Israel Deaconess Medical Center approved the study protocols.

### Measurements

We obtained data directly from participants and from medical records, as detailed previously (Yunginger et al., Am. Rev. Respir. Dis. 146:888-894, 1992). Briefly, maternal diet assessment at both the 1^{st} and 2^{nd} visits was based on a validated 166-item semi-quantitative food frequency questionnaire (FFQ) (Fawzi et al., Ann. Epidemiol. 14:754-762, 2004). We slightly modified the FFQ for use in pregnancy from the FFQ used in the Nurses' Health Study and other cohort studies (Willett, New York: Oxford University Press; 1998). Modifications for use in pregnancy included changing the time referent, beverage section, and vitamin and supplement assessment. The FFQ used at the 1^{st} visit reflected intakes in the 1^{st} trimester and the time referent was “during this pregnancy.” In addition, we collected information on beverage intake during 2 specified time periods: before and after the participant learned that she was pregnant. To assess vitamin and supplement intake during the 1^{st} trimester, we administered a separate interview that assessed dose, duration, and brand/type of multivitamin, prescribed prenatal vitamin, and supplements. The FFQ used at the 2^{nd} visit (26 to 28 weeks of gestation) reflected intakes during the 2^{nd} trimester; the time referent was “during the past 3 months.” The 2^{nd} trimester instrument was the same as the 1^{st} trimester, except that we assessed use of vitamins/supplements as part of the self-completed FFQ and we collected beverage information during the one time period.

To calculate intake of nutrients, we used the Harvard nutrient composition database (Hu, N. Engl. J. Med. 337:1491-1499, 1997). We calculated vitamin D intake for each FFQ, and the mean of these two values was the assigned exposure for each woman during pregnancy (Rifas-Shiman, Paediatr. Perinat. Epidemiol. 20:35-42, 2006). We used a similar approach for retinol and calcium intakes. For analyses based on foods rather than nutrients, we focused on milk (servings/day), the major contributor to vitamin D intake in the diet. We also examined foods with possible effects on asthma risk (McKeever et al., Am. J. Respir. Crit. Care Med. 170:725-729, 2004; Devereux et al., J. Allergy Clin. Immunol. 115:1109-1117, 2005): fruits and vegetables (servings/day) and fish (servings/week).

At the 1^{st} visit in early pregnancy, in addition to diet assessment, we obtained information on maternal age, race/ethnicity, household income, education, marital status, last menstrual period (LMP), height, pre-pregnancy weight, gravidity, and history of atopic disorders. At the 2^{nd} visit in mid-pregnancy, we updated many of these variables. We calculated maternal pre-pregnancy body-mass index as weight in kilograms divided by the square of height in meters. We calculated gestational age using the mother's reported last menstrual period, or from second trimester ultrasound if they differed by more than 10 days. From the hospital vital statistics record, we abstracted infant birth weight. We determined birth weight for gestational age z-value (“fetal growth”) by use of U.S. national reference data (Oken et al., BMC Pediatr. 3:6, 2003). At the 6-month and 1-year visits, we asked about infant feeding, vitamin supplements, and household exposures. At the 2-year visit, we also used a validated FFQ to assess children's vitamin D intake (Blum et al., Matern. Child Health J. 3:167-172, 1999).

Our primary outcome was based on the Asthma Predictive Index (Castro-Rodriguez et al., Am. J. Respir. Crit. Care Med. 162:1403-1406, 2000). We assessed wheeze status initially using two questions on each of the three annual visits: 1) “since your child was born (was 12 months old, was 2 years old) has he/she ever had wheezing (or whistling in the chest),” and 2) “since your child was born (was 12 months old, was 2 years old), how many attacks or episodes of wheezing has he/she had?” We defined “recurrent wheeze” as 2 or more wheezing attacks (summed from the 1-, 2-, and 3-year annual questionnaires) among children with a parental history of asthma or personal diagnosis of eczema.

Secondary wheeze outcomes included the larger group of children with “any wheeze” (i.e., any report of wheezing during the first three years of life). Also, at the other end of the wheezing spectrum, we looked at two subsets of children with recurrent wheeze: 1) those who also had a “doctor-diagnosis of asthma” (by maternal report); and 2) those with “4 or more wheezing attacks” (summed over the annual questionnaires). These two subgroup analyses excluded subjects with an intermediate phenotype so that children who satisfied the more stringent case definitions were compared to children without any wheeze.

Finally, we examined two related outcomes: respiratory infection and eczema. We defined “respiratory infection” as a positive response to at least one of three questions on any of the annual questionnaires: “since your child was born (was 12 months old, was 2 years old), have you been told by a health professional that he/she has: (a) bronchiolitis; (b) pneumonia; (c) bronchitis, croup, or any other respiratory infection?” We defined “eczema” as a positive response to one question on any of the annual questionnaires: “have you ever been told by a health care professional that your child has eczema?”

### Data Analysis

To assess the associations between vitamin D intake and clinical outcomes, we used multiple logistic regression models. We assessed the confounding effect of covariates by examining the association of vitamin D intake with outcomes before and after adding the covariates to the model. From the models, we present results using an odds ratio (OR) and 95% confidence interval (CI). We calculated p for trend across quartiles using median values within each quartile. We examined effect modification of the association of maternal intake of vitamin D with risk of recurrent wheeze by six factors: pre-pregnancy BMI (dichotomized at median of 23.4 kg/m2), maternal calcium intake during pregnancy (1200 mg/day), maternal retinol intake during pregnancy (2500 IU/day), parental history of asthma (yes/no), white maternal race (yes/no), and season of LMP. We first put an interaction term in the model (continuous total vitamin D×factor), and then ran multivariate models stratified by the factor. We performed all analyses using SAS version 8.2 (SAS Institute, Cary, N.C.).

The mean (SD) age of mothers at enrollment was 32.5 (4.9) years. Most mothers (72%) had at least college education and 65% lived in households with an annual income of >$70,000; Mean pre-pregnancy BMI was 24.5 (5.1) and 10% of mothers smoked during the index pregnancy. Approximately half of the children were male (51%) and 74% were white. Their mean birthweight was 3.51 (0.52) kg, with a mean gestational age of 39.6 (1.5) weeks. Children were breastfed for an average of 6.4 (4.5) months, and 53% were born into homes with other children less than 12 years old. Among the mothers, 31% had either asthma (17%) or eczema (20%), while among the fathers, 26% had either asthma (14%) or eczema (15%).

The mean (SD) total vitamin D intake during pregnancy was 548 (167) IU/day, with an average of 225 IU from food and 319 IU from supplements. Maternal intake of vitamin D during pregnancy was less than 400 IU/day in 19% of women. Milk was the primary food contributor to vitamin D intake during pregnancy, accounting for 53% of intake. Other substantial contributors for mothers were fish (18%) and cold cereal (9%).

Maternal vitamin D intake was associated with several factors that might affect risk of asthma (Table 1). Mothers with higher intake of vitamin D were slightly older, less overweight, of higher socioeconomic status, less likely to smoke during pregnancy, and more likely to have a personal history of eczema. They consumed a little more fish but their intake of fruits and vegetables did not differ. Maternal vitamin D intake was not associated with conception during the winter months (i.e., fall birthday), gender, birth weight, or gestational age. However, children of women with higher maternal intake of vitamin D were more likely to be born to white mothers, have been breastfed longer, to take a vitamin supplement in the first 6 months of life, and to consume more vitamin D from foods at age 2 years. Among children, the mean (SD) vitamin D intake from foods was 249 (101) IU/day; milk contributed 71% of vitamin D, fish 1%, and cold cereal 17%. The home environment of mothers with high vitamin D intake was characterized by less passive smoke exposure, fewer siblings <age 12, but no difference in exposure to pets or other common allergens.

TABLE 1
Characteristics of 1,194 mother-infant pairs in Project Viva,
according to maternal intake of vitamin D during pregnancy¹

| | Quartile of Vitamin D Intake during Pregnancy | | | | |
|---|---|---|---|---|---|
| | 1 | 2 | 3 | 4 | p-value |
| Median intake (IU) | 356 | 513 | 603 | 724 | |
| Range | 60-445 | 446-562 | 563-658 | 659-1,145 | |
| Mother and Family History: | | | | | |
| Maternal age at enrollment | 31.3 (5.7) | 32.6 (4.6) | 32.7 (4.5) | 33.5 (4.4) | <0.001 |
| (years) | | | | | |
| Pre-pregnancy body mass index | 25.7 (6.0) | 24.2 (4.9) | 24.1 (4.5) | 24.0 (4.6) | <0.001 |
| (kg/m²) | | | | | |
| Maternal Dietary Intake: | | | | | |
| Vitamin D from foods (IU) | 164 (76) | 186 (78) | 228 (75) | 320 (95) | <0.001 |
| Vitamin D from supplements (IU) | 163 (107) | 322 (86) | 370 (67) | 421 (86) | <0.001 |
| Calcium intake (mg) | 1109 (284) | 1327 (301) | 1447 (299) | 1620 (335) | <0.001 |
| Retinol equivalents of Vitamin | 1663 (579) | 2087 (577) | 2328 (572) | 2582 (692) | <0.001 |
| A (mcg) | | | | | |
| Fruit and vegetables | 5.8 (2.9) | 5.9 (2.4) | 6.1 (2.5) | 6.0 (3.1) | 0.17 |
| (servings/day) | | | | | |
| Fish (servings/week) | 1.7 (1.4) | 1.8 (1.3) | 1.9 (1.4) | 2.1 (1.7) | <0.001 |
| College graduate (%) | 53 | 74 | 80 | 82 | <0.001 |
| Household income of >$70,000/year (%) | 55 | 66 | 68 | 71 | <0.001 |
| Mother smoked during index | 13 | 12 | 9 | 6 | 0.001 |
| pregnancy (%) | | | | | |
| Family History: | | | | | |
| Maternal asthma (%) | 19 | 16 | 21 | 13 | 0.09 |
| Maternal eczema (%) | 15 | 21 | 18 | 24 | 0.02 |
| Maternal asthma or eczema (%) | 30 | 30 | 32 | 31 | 0.62 |
| Paternal asthma (%) | 18 | 17 | 9 | 14 | 0.05 |
| Paternal eczema (%) | 15 | 17 | 13 | 14 | 0.38 |
| Paternal asthma or eczema (%) | 30 | 28 | 21 | 25 | 0.05 |

Child and Home Environment:

| | | | | | |
|---|---|---|---|---|---|
| Maternal LMP during winter (%) | 18 | 21 | 24 | 23 | 0.08 |
| Boy (%) | 50 | 52 | 48 | 53 | 0.68 |
| White (%) | 63 | 78 | 78 | 79 | <0.001 |
| Birth weight (kg) | 3.48 (0.52) | 3.53 (0.55) | 3.53 (0.48) | 3.51 (0.51) | 0.54 |
| Gestational age at birth (weeks) | 39.5 (1.5) | 39.6 (1.5) | 39.8 (1.4) | 39.6 (1.4) | 0.31 |
| Fetal growth (birth weight for | 0.16 (1.0) | 0.25 (1.0) | 0.19 (0.9) | 0.20 (0.9) | 0.72 |
| GA z-score) | | | | | |
| Dietary Intake During Childhood: | | | | | |
| Breastfeeding for >=9 months | 32 | 37 | 41 | 49 | <0.001 |
| (%) | | | | | |
| Number of months | 5.7 (4.5) | 6.0 (4.6) | 6.7 (4.5) | 7.3 (4.4) | <0.001 |
| breastfeeding | | | | | |
| Vitamin supplement in first 6 | 10 | 15 | 18 | 20 | <0.001 |
| months (%) | | | | | |
| Vitamin D intake from foods at | 225 (108) | 248 (103) | 251 (85) | 267 (106) | <0.001 |
| age 2 (IU) | | | | | |
| Passive smoke exposure at home | 14 | 6 | 5 | 6 | <0.001 |
| (%) | | | | | |
| Number of children <12 years at | 1.8 (0.8) | 1.7 (0.8) | 1.6 (0.7) | 1.6 (0.7) | <0.001 |
| home | | | | | |
| Furry pet at home (%) | 40 | 42 | 42 | 41 | 0.80 |
| Home cockroach exposure (%) | 4 | 4 | 4 | 3 | 0.50 |
| Moisture or mildew in home (%) | 28 | 28 | 27 | 30 | 0.82 |

¹Unless otherwise indicated, results are mean (standard deviation). IU denotes international units; LMP, mother's last menstrual cycle; and GA, gestational age).

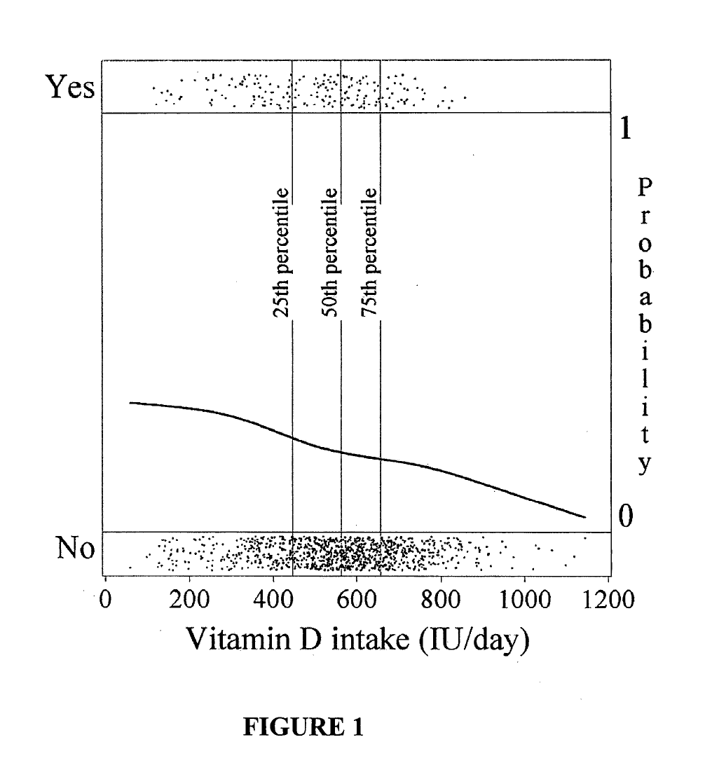

FIG. 1 shows the unadjusted inverse linear association between maternal vitamin D intake and risk of recurrent wheeze in offspring. Since individual observations would otherwise be difficult to distinguish, we added random noise to the display of each observation. The smoothed line shows the approximate probability of recurrent wheeze for each observed value of vitamin D intake. Table 2 confirms this strong inverse association (p for trend <0.001). Compared with mothers in the lowest quartile of daily intake (median within quartile: 356 IU), those in the highest quartile (median: 724 IU) had lower risk of a child with recurrent wheeze at age 3 (OR 0.39; 95% CI, 0.25-0.62). The strong inverse association between maternal vitamin D intake and recurrent wheeze was robust to controlling for a variety of potential confounders, including traditional asthma risk factors and putative dietary risk factors such as fish, fruits, and vegetables. By contrast, maternal intake of vitamin D did not affect risk of respiratory infections or early childhood eczema (Table 2). Multivariate model 1 adjusts for sex, birth weight, income, maternal age, maternal pre-pregnancy body mass index, passive smoking exposure, breastfeeding duration at one year, number of children less than 12 years of age in household, maternal history of asthma, and paternal history of asthma. For eczema analyses, the model adjusts for parental history of eczema instead of asthma. Multivariate model 2 adjusts for the 10 factors above plus intake of fish and fruits and vegetables.

TABLE 2
Association between maternal intake of vitamin D during pregnancy and risk of
clinical outcomes in children at age 3 years (n = 1,194 mother-infant pairs)

| | Quartile of Vitamin D Intake by Mother during Pregnancy | | | | |
|---|---|---|---|---|---|
| | 1 | 2 | 3 | 4 | p-value |
| Median intake (IU) | 356 | 513 | 603 | 724 | |
| Range | 60-445 | 446-562 | 563-658 | 659-1,145 | |
| n | 298 | 299 | 299 | 298 | |
| Recurrent wheeze | | | | | |
| (n = 186 cases) | | | | | |
| Unadjusted | 1.00 | 0.55 | 0.55 | 0.39 | <0.001 |
| | | (0.36, 0.84) | (0.36, 0.83) | (0.25, 0.62) | |
| Multivariate model 1 | 1.00 | 0.49 | 0.56 | 0.41 | 0.001 |
| | | (0.30, 0.80) | (0.34, 0.92) | (0.24, 0.70) | |
| Multivariate model 2 | 1.00 | 0.47 | 0.54 | 0.38 | <0.001 |
| | | (0.28, 0.77) | (0.33, 0.89) | (0.22, 0.65) | |
| Respiratory infection | | | | | |
| (n = 540 cases) | | | | | |
| Unadjusted | 1.00 | 0.68 | 0.92 | 0.68 | 0.09 |
| | | (0.48, 0.96) | (0.65, 1.30) | (0.48, 0.96) | |
| Multivariate model 1 | 1.00 | 0.73 | 1.01 | 0.74 | 0.28 |
| | | (0.51, 1.06) | (0.70, 1.45) | (0.51, 1.08) | |
| Multivariate model 2 | 1.00 | 0.74 | 1.01 | 0.75 | 0.32 |
| | | (0.51, 1.06) | (0.70, 1.46) | (0.52, 1.09) | |
| Eczema | | | | | |
| (n = 428 cases) | | | | | |
| Unadjusted | 1.00 | 1.00 | 1.03 | 1.10 | 0.58 |
| | | (0.71, 1.39) | (0.74, 1.44) | (0.79, 1.53) | |
| Multivariate model 1 | 1.00 | 0.88 | 0.94 | 0.95 | 0.88 |
| | | (0.60, 1.27) | (0.65, 1.37) | (0.65, 1.39) | |
| Multivariate model 2 | 1.00 | 0.86 | 0.93 | 0.92 | 0.75 |
| | | (0.59, 1.25) | (0.64, 1.36) | (0.63, 1.35) | |

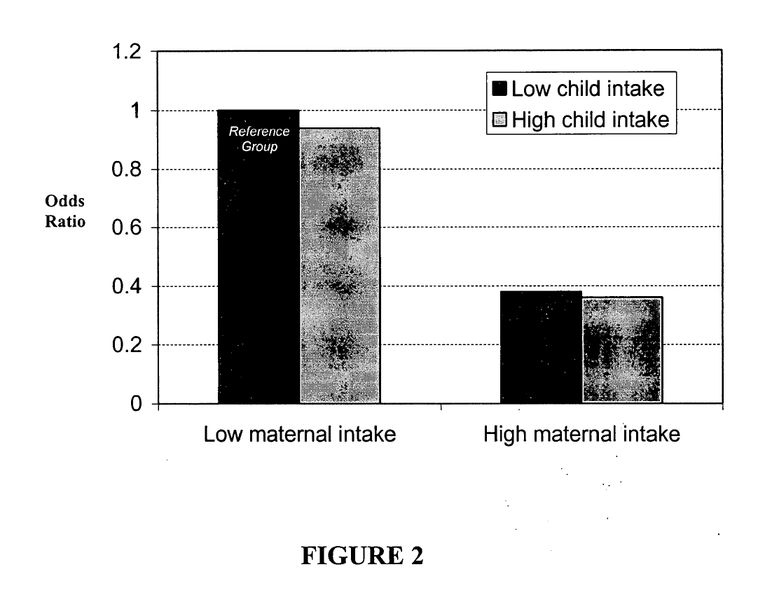

To further explore these results, we examined risk according to vitamin D intake both during pregnancy and over the first two years of life. As shown in Table 1, women with higher vitamin D intake during pregnancy were more likely to have children who used vitamin supplements and ate more foods with vitamin D. Nevertheless, the association of maternal vitamin D intake with recurrent wheeze (Table 2, model 2) did not change with further control for child use of a vitamin supplement at age 6 months (OR 0.39; 95% CI, 0.23-0.67; p for trend <0.001) or for child vitamin D intake from foods at 2 years (OR 0.34; 95% CI, 0.20-0.60; p for trend <0.001). FIG. 2 examines this issue in another way: stratifying mothers and children by the vitamin D intake of each group, with high-low cutpoints set at 400 IU for mothers and 200 IU for children. The figure demonstrates that the decreased risk of recurrent wheeze was largely due to high maternal intake of vitamin D during pregnancy.

Table 3 shows risk of recurrent wheeze with maternal vitamin D intake expressed as a 100 IU/day increase rather than in categories. The results closely resemble the strong inverse association in Table 2. Moreover, results were similar for vitamin D from food only and vitamin D from supplements only. Further adjustment for race/ethnicity and college education did not change the inverse association between maternal vitamin D intake and risk of recurrent wheeze (OR 0.81. 95% CI, 0.72-0.90). We also examined the potential impact of two dietary factors correlated with vitamin D intake: retinol (r=0.53) and calcium (r=0.57). Neither nutrient confounded the strong inverse association between maternal vitamin D intake and risk of recurrent wheeze (Table 3). To further explore the spectrum of wheezing illnesses of childhood, we looked at the relation of maternal vitamin D intake with risk of any wheeze (n=416 cases) and with subsets of the primary outcome: recurrent wheeze with doctor-diagnosis of asthma (n=132 cases), and recurrent wheeze with 4+ wheezing episodes (n=86 cases). For each 100 IU increase of maternal vitamin D intake, we observed a somewhat weaker but still significantly lower risk of any wheeze (OR 0.89; 95% CI, 0.82-0.97). The inverse association appeared stronger for the most stringent wheeze outcomes: children with recurrent wheeze and a doctor-diagnosis of asthma (OR 0.82; 95% CI, 0.71-0.93) or recurrent wheeze with 4+ wheezing episodes (OR 0.79; 95% CI, 0.67-0.93). All of these values were adjusted for the 12 factors in model A (Table 3). Multivariate model A adjusts for 12 factors: sex, birth weight, income, maternal age, maternal pre-pregnancy body mass index, passive smoking exposure, breastfeeding duration at one year, number of children less than 12 years of age in household, maternal history of asthma, paternal history of asthma, fish intake, and intake of fruits and vegetables. Multivariate model B adjusts for the 12 factors above plus maternal intake of vitamin D from supplements (in food only models) and for vitamin D from foods (in supplement only models). Multivariate model C adjusts for the 12 factors in model A plus maternal intake of calcium. Multivariate model D adjusts for the 12 factors in model A plus maternal intake of retinol.

TABLE 3
Association between maternal intake of vitamin D during
pregnancy and risk of recurrent wheeze in children at age
3 years (n = 1,194 mother-infant pairs)

| | Vitamin D Intake (per 100 IU/day) | | |
|---|---|---|---|
| | Total | Food Only | Supplement Only |
| Unadjusted | 0.81 | 0.81 | 0.82 |
| | (0.74, 0.89) | (0.69, 0.96) | (0.73, 0.92) |
| Multivariate model A | 0.80 | 0.79 | 0.82 |
| | (0.72, 0.90) | (0.65, 0.96) | (0.71, 0.95) |
| Multivariate model B | — | 0.80 | 0.82 |
| | | (0.66, 0.97) | (0.71, 0.95) |
| Multivariate model C | 0.79 | — | — |
| | (0.69, 0.91) | | |
| Multivariate model D | 0.81 | — | — |
| | (0.71, 0.93) | | |

As noted earlier, a major dietary source of vitamin D is fortified milk. Accordingly, we examined the association between milk intake by mothers during pregnancy and risk of clinical outcomes in their children. Compared with mothers who did not drink milk (who had a median vitamin D intake of 479 IU from other foods and supplements), and after adjusting for 12 factors, risk of recurrent wheeze was lowest among those drinking 1 to 1.9 cups/day (OR 0.35; 95% CI, 0.16-0.80). The associations were less extreme for those drinking <1 cup/day (OR 0.57; 95% CI, 0.27-1.24) or 2+ cups/day (0.45; 95% CI, 0.20-1.02), and the p for linear trend was 0.13.

Finally, we explored effect modification of the association of maternal intake of vitamin D with risk of recurrent wheeze. Only season of LMP demonstrated a likely interaction (p=0.06). Maternal intake of vitamin D was associated with a lower risk of recurrent wheeze when the LMP was in the winter. The multivariate OR per 100 IU/day increase was 0.62 (95% CI 0.47-0.83) for LMP in winter versus 0.85 (95% CI 0.75-0.97) in other seasons.

In summary, we found a strong inverse association between maternal intake of vitamin D during pregnancy and risk of recurrent wheeze in children at age 3 years. The inverse association was independent of many potential confounders, including adjustment for fish, fruits and vegetables, early use of vitamin supplement, and child intake of vitamin D at age 2 years. The association was present for vitamin D from either foods or nutritional supplements. In addition, among children conceived in the winter months and therefore born in the fall (a time of low sunlight exposure), the inverse association between maternal intake of vitamin D and risk of recurrent wheeze was stronger. We note that the early age of outcome measurements limits our ability to generalize these findings from recurrent wheeze to actual asthma. Many children who wheeze before age 3 have transient episodes and do not go on to have asthma (Martinez et al., N. Engl. J. Med. 332:133-138, 1995). However, the chosen 3-year outcome carries an almost 10-fold increased risk of developing asthma by age 6 (Castro-Rodriguez et al., Am. J. Respir. Crit. Care Med. 162:1403-1406, 2000). Thus, the present data provide a reliable basis for making a correlation between vitamin D intake and asthma.

### Project Viva: Antioxidant Study

### Design and Participants

Study subjects were enrolled in Project Viva, as described above. Recruitment and retention procedures have previously been published (Gillman et al., J. Pediatr. 144:240-245, 2004). We obtained information directly from participants and from medical records as detailed previously (Rifas-Shiman, Paediatr. Perinat. Epidemiol. 20:35-42, 2006). Data collected from interviews and questionnaires included demographics (infant gender, maternal marital status, and race), socioeconomic status (education and household income), and reproductive history (maternal gravidity, pre-pregnancy height and body mass index, and smoking). Questionnaires that inquired about infant health, feeding, and the home environment were administered when the child was 6 months, 1 year, and 2 years of age. Among 2,128 delivered infants in Project Viva, we excluded 228 participants because of missing first and second trimester diet assessment data and an additional 37 participants with a gestation of less than 34 completed weeks. Mothers of 368 of the remaining 1863 children either did not provide informed consent for child follow-up through age 2 years, or missed the 2-year follow-up questionnaire. Of the remaining 1495, 181 were lost to follow-up, and 24 did not yet exit the 2 year time-window at the time of this analysis, leaving a sample for analysis of 1,290 participants.

### Dietary Assessment

We assessed maternal diet using semi-quantitative food-frequency questionnaires (FFQ) validated for use during pregnancy (Fawzi et al., Ann. Epidemiol. 14:754-762, 2004), and used in several of our previous studies of maternal diet and offspring outcomes (Oken et al., Am. J. Epidemiol. 160:774-783, 2004; Huh et al., Int. J. Epidemiol. 34:378-384, 2005; Gillman et al., Circulation 110:1990-1995, 2004). As noted above, the FFQ used at the 1^{st} visit reflected intakes in the 1^{st} trimester; the time referent was “during this pregnancy,” that is from the date of the last menstrual period until the assessment, at an average of about 10 weeks gestation. To assess vitamin and supplement intake during the 1^{st} trimester, we administered a separate interview that queried dose, duration, and brand/type of multivitamin, prescribed prenatal vitamins, and supplements. Also as noted above, the FFQ used at the 2^{nd} visit (26 to 28 weeks of gestation) reflected intakes during the 2^{nd} trimester; the time referent was “during the past 3 months.” The 2^{nd} trimester instrument was the same as that for the 1^{st} trimester except that we assessed use of vitamins/supplements as part of the self-completed FFQ.

To calculate intake of nutrients, we again used the Harvard nutrient composition database, which contains food composition values from the U.S. Department of Agriculture, supplemented by other data sources (U.S. Department of Agriculture ARS. USDA National Nutrient Database for Standard Reference, Release 14, 5R14; 2001). All nutrients were adjusted for total energy intake using the nutrient residuals method (Willett, Am. J. Clin. Nutr. 45:354-360, 1987). Mean nutrient intakes from the 1^{st} and 2^{nd} trimesters were used as the exposure in the analyses. If a participant only completed one FFQ, then nutrients calculated from that questionnaire were taken as the exposure of interest. One thousand one hundred and thirty-five (88.0%) of the 1,290 participants completed both FFQs, 96 (7.4%) completed the first trimester FFQ only, and 59 (4.6%) completed the second trimester FFQ only.

### Definition of 2-Year Outcome Variables

We ascertained the wheeze outcomes from questions from the First Year and Second Year Questionnaires: “since your child was born/since your child was 12 months old, has he/she ever had wheezing (or whistiling in the chest)?” “Any wheeze” was defined as a positive response to the question at either the first or second year questionnaire. “Recurrent wheeze” was defined as positive responses to the questions in both the first and second year questionnaires. If the child had a positive response to either, but a negative response on the other questionnaire, he/she was excluded from the analysis for recurrent wheeze, making the comparison group for both wheeze outcomes the children who never wheezed in the first 2 years of life. “Respiratory infections” was defined as positive responses to one of three questions: “since your child was born/12 months old, have you been told by a health care professional (such as a doctor, physician assistant, or nurse practitioner) that your child has: (a) bronchiolitis; (b) pneumonia; (c) bronchitis, croup, or any other respiratory infection?” on either the first or second year questionnaires. “Eczema” was defined as a positive response to the question: “have you ever been told by a health care professional (such as a doctor, physician assistant, or nurse practitioner) that your child has eczema?” on either the first or second year questionnaires.

### Demographics, Birth, Parental Conditions, and Other Variables

Parental demographic and health history information was collected by interview and self-administered questionnaires (Gillman et al., J. Pediatr. 144:240-245, 2004). Infant birth weight data were obtained from hospital records. Multivitamin intake in the first year was determined by the questions at the 6-month and 1-year questionnaires. At 6 months, mothers were asked “since your baby was born, has your baby taken any multivitamin drops such as Tri-Vit (Tri-Vi-Sol) or Poly-Vi-Sol? Other vitamins or supplements?” At 1 year, mothers were asked “in the past month, has your child taken any of the following vitamins or supplements? Multivitamin drops such as Tri-Vit (Tri-Vi-Sol) or Poly-Vi-Sol (Yes/No); Other vitamins or supplements?” Duration of breastfeeding was ascertained from detailed questions at the 1-year questionnaire.

### Statistical Analysis

We examined the associations of each nutrient with each outcome using bivariate logistic regression models. Nutrients that had statistically significant associations with either wheeze or eczema outcome in the bivariate logistic regression models were then taken forward to multivariable models. To assess the multivariable association between maternal antioxidant intake with the 2-year outcomes, we first performed bivariate analyses to determine the non-nutrient characteristics associated with these outcomes, using logistic regression. Variables that were significantly associated (p<0.05) with any of the 3 outcomes were included in the respective multivariable model. For the wheeze and the respiratory infections, these variables included birth weight, infant gender, maternal age, maternal pre-pregnancy BMI, breastfeeding duration, the number of children under the age of 12 years in the home, post-natal passive smoke exposure, family income, and maternal and paternal asthma. For eczema, maternal and paternal asthma were replaced with maternal and paternal eczema. Additionally, models for the individual nutrients and individual non-nutrient variables were created to test for any potential confounders of the relation between nutrient and outcome. We defined a confounder as a variable that causes more than an 8% change in the estimate when entered into the model. None of the non-nutrient variables caused a change of more than 8% in the estimate for the antioxidant nutrient in these models. Thus, no other variable aside from those listed above were included in the multivariable analyses. Additional multivariable models were created to adjust for maternal intakes of fruits and vegetables (since these are main sources of antioxidants in the diet) and the child's intake of multivitamins in the first year (since this is a potential confounder of the maternal diet-infant outcome relationship).

To determine the most parsimonious models, variables that were not statistically significant in the full multivariable models were removed and the subsequent models were compared with the full models using likelihood ratio tests. Statistical analyses were performed using SAS statistical software, version 8.2 (SAS Institute, Inc., Cary, N.C.).

### Results

The characteristics of the participants are presented in Table 4. Three hundred seventy-six (32.4%) children had any wheeze in the first 2 years of life, while 136 (13.1%) children had recurrent wheezing. Four hundred ten (31.9%) children had eczema. Fifty one percent of the children were male and 75.6% were white. The mean age of the mothers on entry into the cohort was 32.6 years, 73.2% of the mothers had at least a college education, 62.6% of the mothers lived in households with an annual income of >$70,000, and only 9.5% of the mothers smoked during pregnancy. Among the mothers, 16.4% had asthma and 19.6% had eczema, while among the fathers, 14.5% had asthma and 14.3% had eczema. A comparison of subjects in the analysis and those who were excluded is also presented in Table 4. Since we excluded premature infants from this current analysis, subjects in the cohort had higher birthweights than excluded subjects. Furthermore, excluded subjects had higher proportions of minority ethnic groups, younger mothers, subjects with shorter duration of breastfeeding, low family income subjects, subjects with low educational attainment, and smoking mothers. There were no differences in the proportions of maternal asthma and paternal asthma between the two groups. With regard to parental eczema, the proportion of maternal and paternal eczema was lower in the group that was excluded from this analysis.

TABLE 4
Participant characteristics

| | | Excluded | |
|---|---|---|---|
| | In Analysis | Subjects¹ | |
| Characteristics | n = 1290 | n = 838 | p-value |
| Child's Gender, n (%) | | | |
| male | 658 (51.0) | 438 (52.3) | 0.57 |
| female | 632 (49.0) | 400 (47.7) | |
| Birthweight, kg, mean (sd) | 3.53 (0.51) | 3.35 (0.68) | <0.0001 |
| Race/ethnicity, n (%) | | | |
| White | 975 (75.6) | 424 (52.0) | |
| Black | 134 (10.4) | 214 (26.3) | |
| Hispanic | 69 (5.4) | 85 (10.4) | <0.0001 |
| Asian | 63 (4.9) | 57 (7.0) | |
| Other | 48 (3.7) | 35 (4.3) | |
| Number of other children in the | | | |
| home under 12 yr, n (%) | | | |
| >1 | 675 (53.9) | 256 (50.8) | 0.24 |
| Breastfeeding duration, months, | | | |
| n (%) | | | |
| None | 139 (11.6) | 84 (23.1) | |
| >0 to <4 | 269 (22.5) | 152 (41.9) | <0.0001 |
| 4 to <10 | 387 (32.3) | 90 (24.8) | |
| 10+ | 402 (33.6) | 37 (10.2) | |
| Multivitamin intake in the first | | | |
| year, n (%) | | | |
| yes | 239 (19.1) | 60 (11.9) | 0.0003 |
| Eczema in the first 2 years of life, | | | |
| n (%) | | | |
| yes | 410 (31.9) | —² | |
| no | 875 (68.1) | | |
| Any wheeze at 2 yr, n (%) | | | |
| yes | 376 (32.4) | —² | |
| no | 785 (67.6) | | |
| Recurrent wheeze at 2 yr, n (%) | | | |
| yes | 136 (13.1) | —² | |
| no | 902 (86.9) | | |
| Lower respiratory tract infection | | | |
| at 2 yr, n (%) | | | |
| yes | 327 (25.7) | —² | |
| no | 944 (74.3) | | |
| Maternal age, yrs, mean (sd) | 32.6 (4.8) | 30.6 (5.6) | <0.0001 |
| Maternal pre-pregnancy BMI, | 24.5 (5.1) | 25.5 (6.2) | <0.0001 |
| mean (sd) | | | |
| Highest educational level, n (%) | | | |
| High school or some college | 346 (26.8) | 398 (48.8) | |
| College graduate | 491 (38.1) | 252 (30.9) | <0.0001 |
| Post graduate degree | 452 (35.1) | 165 (20.3) | |
| Meternal smoking during | 122 (9.5) | 133 (15.9) | <0.0001 |
| pregnancy, n (%) | | | |
| Passive smoke exposure in the | 0.26 (1.31) | 0.20 (1.02) | 0.32 |
| home, hours/week, mean (sd) | | | |
| Household income, n (%) | | | |
| <=$40,000 | 139 (10.8) | 153 (18.3) | |
| >$40,000 to $70,000 | 278 (21.6) | 156 (18.6) | <0.0001 |
| >$70,000 | 807 (62.6) | 337 (40.2) | |
| missing | 66 (5.1) | 192 (22.9) | |
| Maternal Asthma | | | |
| yes | 211 (16.4) | 137 (16.4) | |
| no | 1079 (83.6) | 701 (83.7) | 0.996 |
| Maternal Eczema | | | |
| yes | 253 (19.6) | 116 (13.8) | 0.0006 |
| no | 1037 (80.4) | 722 (86.2) | |
| Paternal Asthma | | | |
| yes | 187 (14.5) | 102 (12.2) | 0.13 |
| no | 1103 (85.5) | 736 (87.8) | |
| Paternal Eczema | | | |
| yes | 184 (14.3) | 50 (6.0) | <0.0001 |
| no | 1106 (85.7) | 788 (94.0) | |

¹Numbers do not always add up to 838 because of variable numbers of missing data for each variable
²There were not enough numbers with information to make valid comparisons. Most had missing information for these variables.

Table 5 presents the distributions of maternal intakes of nutrients. For most of the nutrients, there was a wide distribution of intake, particularly when supplements were included. Most of the women met the recommended minimum daily requirements for folate (400 mcg/day) (Proceedings of the 1992 International Symposium on Public Health Surveillance, Atlanta, Ga., Apr. 22-24, 1992. MMWR Morb. Mortal. Wkly. Rep. 41, 1992. ACOG practice bulletin, Obstet. Gynecol. 102(44):203-213, 2003), zinc (15 mg) (Institute of Medicine, Food and Nutrition Board, Committee on Nutritional Status During Pregnancy, Washington, D.C.: National Academy Press, 1990), vitamin C (85 mg/day) (Institute of Medicine, Food and Nutrition Board, Panel on Dietary Antioxidants and Related Compounds, Subcommittees on Upper Reference Levels of Nutrients and Interpretation and Uses of DRIs, Standing Committee on the Scientific Evaluation of Dietary Reference Intakes: Washington, D.C.: National Academy Press, 2000), and vitamin E (15 mg/day) (Institute of Medicine, Food and Nutrition Board, Panel on Dietary Antioxidants and Related Compounds, Subcommittees on Upper Reference Levels of Nutrients and Interpretation and Uses of DRIs, Standing Committee on the Scientific Evaluation of Dietary Reference Intakes: Washington, D.C.: National Academy Press, 2000), when total intakes (foods+supplements) were considered. However, if antioxidant intakes from foods only are considered, more than half of the women would not have met the recommended minimum daily requirements for both folate and zinc.

TABLE 5
Maternal nutrient intake during pregnancy¹

| | | | 25th | | 75th | |
|---|---|---|---|---|---|---|
| Nutrient² | Mean ± SD | Minimum | percentile | Median | percentile | Maximum |
| Vitamin C, mg | 279.6 ± 92.1 | 47.3 | 207.0 | 254.3 | 313.3 | 3925.6 |
| Vitamin C w/out suppl, | 174.3 ± 64.8 | 35.0 | 129.1 | 167.3 | 210.8 | 617.5 |
| mg | | | | | | |
| Vitamin E, mg | 30.7 ± 64.6 | 3.6 | 14.3 | 18.8 | 26.5 | 1202.2 |
| Vitamin E w/out suppl, | 8.4 ± 6.5 | 2.9 | 5.5 | 6.4 | 8.1 | 67.9 |
| mg | | | | | | |
| α Carotene, mcg | 878.0 ± 657.7 | 3.9 | 425.8 | 732.0 | 1136.7 | 5221.7 |
| B Carotene, mcg | 4770.3 ± 2293.0 | 323.3 | 3279.7 | 4403.3 | 5875.5 | 36744.5 |
| β Carotene w/out suppl, | 3863.5 ± 2036.5 | 325.0 | 2433.3 | 3496.3 | 4925.5 | 16,553.3 |
| mcg | | | | | | |
| B Cryptoxanthin, mcg | 207.3 ± 130.9 | 0.5 | 112.6 | 181.5 | 272.9 | 1207.2 |
| Folate, mcg | 1117.6 ± 422.3 | 148.8 | 917.3 | 1157.5 | 1327.4 | 10220.1 |
| Folate w/out suppl, mcg | 368.4 ± 112.9 | 146.7 | 289.7 | 352.9 | 425.6 | 1196.9 |
| Lutein + Zeaxanthin, | 2686.8 ± 1724.4 | 133.0 | 1570.5 | 2286.3 | 3317.8 | 14069.6 |
| mcg | | | | | | |
| Lycopene, mcg | 7368.8 ± 3979.7 | 489.3 | 4730.2 | 6649.8 | 9273.6 | 33889.0 |
| Copper, mg | 1.6 ± 0.5 | 0.8 | 1.3 | 1.5 | 1.7 | 8.8 |
| Copper w/out suppl, mg | 1.4 ± 0.3 | 0.8 | 1.3 | 1.4 | 1.6 | 2.7 |
| Zinc, mg | 30.3 ± 9.3 | 6.8 | 25.2 | 31.5 | 36.0 | 92.0 |
| Zinc w/out suppl, mg | 12.4 ± 3.3 | 6.4 | 10.6 | 11.8 | 13.3 | 40.2 |

¹Nutrients were calculated from FFQs as described in the Methods section. For each participant, the means of the first and second trimester nutrients were obtained.
²Unless specifically stated, values are for total nutrient intakes (foods + supplements).

We did not find any associations between any of the nutrients and eczema at 2 years of age. Neither maternal intakes of fruits and vegetables nor infant multivitamin intake were independently associated with any of the outcomes. On the other hand, total intakes of vitamin C, vitamin E, zinc, folic acid, lutein+zeaxanthin, β-carotene, and copper were associated with both wheeze outcomes.

Table 6 presents the results of the analyses on the wheeze outcomes at 2 years of age. For any wheeze, total intakes of vitamin C, vitamin E, zinc, folic acid, lutein+zeaxanthin, β-carotene, and copper were all associated with lower risks in univariable models. In a multivariable model adjusted for potential confounders, maternal intakes of zinc in the highest quartile remained protective for any wheeze (OR=0.59, 95% CI=0.41, 0.88; p=0.01), and there was a trend toward a protective effect for maternal intakes of vitamin E in the highest quartile (OR=0.70, 95% CI=0.48, 1.03; p=0.06). Additional adjustment for multi-vitamin intake in the first year of life did not change these associations. For recurrent wheeze, similar findings were noted in univariable models. In multivariable models, maternal intakes in the highest quartile of vitamin E (OR=0.49, 95% CI=0.27, 0.90) and zinc (OR=0.49, 95%0=0.27, 0.87) remained inversely associated with recurrent wheeze (all p<0.05 when compared with lowest quartile of intake). Because most of the antioxidants in the diet come from fruits and vegetables, we ran additional models adjusting for maternal fruits and vegetable intake as a way of adjusting for other antioxidants. The results of these additional models were not different from the models presented in Table 6. Similar results were also obtained when the analysis was stratified by maternal asthma.

TABLE 6
Associations between maternal antioxidant intakes and outcomes at
2 years of age

| | Any wheeze in the | | Recurrent wheeze in | |
|---|---|---|---|---|
| | first 2 yrs n = 1,161 | | the first 2 yrs n = 1,038² | |
| | | Multivariable | | Multivariable |
| Nutrients¹ | Univariate Model | Model³ | Univariate Model | Model⁴ |
| (Quartiles of intake) | OR (95% CI) | OR (95% CI) | OR (95% CI) | OR (95% CI) |
| Vitamin C | | | | |
| 1^{st} (lowest) | 1.00 | 1.00 | 1.00 | 1.00 |
| 2^{nd} | 0.82 (0.58, 1.15) | 0.93 (0.65, 1.35) | 1.02 (0.63, 1.65) | 1.05 (0.62, 1.77) |
| 3^{rd} | 0.65 (0.46, 0.93)⁵ | 0.84 (0.58, 1.22) | 0.85 (0.52, 1.40) | 1.04 (0.60, 1.78) |
| 4^{th} (highest) | 0.67 (0.47, 0.94)⁵ | 0.79 (0.54, 1.15) | 0.56 (0.32, 0.97)⁵ | 0.75 (0.41, 1.36) |
| p for trend⁶ | 0.01 | 0.2 | 0.03 | 0.4 |
| Vitamin E | | | | |
| 1^{st} (lowest) | 1.00 | 1.00 | 1.00 | 1.00 |
| 2^{nd} | 0.71 (0.51, 1.01) | 0.90 (0.62, 1.31) | 0.60 (0.37, 0.98) | 0.79 (0.47, 1.34) |
| 3^{rd} | 0.67 (0.48, 0.95)⁵ | 0.84 (0.58, 1.22) | 0.68 (0.43, 1.10) | 0.90 (0.54, 1.52) |
| 4th (highest) | 0.52 (0.36, 0.74)⁵ | 0.70 (0.48, 1.03) | 0.36 (0.20, 0.62)⁵ | 0.49 (0.27, 0.90)⁵ |
| p for trend⁶ | 0.0004 | 0.06 | 0.0007 | 0.05 |
| Zinc | | | | |
| 1st (lowest) | 1.00 | 1.00 | 1.00 | 1.00 |
| 2^{nd} | 0.77 (0.55, 1.08) | 0.89 (0.61, 1.29) | 0.56 (0.34, 0.92) | 0.61 (0.35, 1.06) |
| 3^{rd} | 0.67 (0.47, 0.94)⁵ | 0.85 (0.59, 1.24) | 0.73 (0.46, 1.17) | 0.89 (0.53, 1.49) |
| 4th (highest) | 0.49 (0.34, 0.70)⁵ | 0.59 (0.41, 0.88)⁵ | 0.41 (0.24, 0.69)⁵ | 0.49 (0.27, 0.87) |
| p for trend⁶ | <.0001 | 0.01 | 0.004 | 0.06 |
| Lutein + zeaxanthin | | | | |
| 1st (lowest) | 1.00 | 1.00 | 1.00 | 1.00 |
| 2^{nd} | 1.00 (0.72, 1.41) | 1.20 (0.83, 1.73) | 1.02 (0.63, 1.67) | 1.35 (0.79, 2.32) |
| 3^{rd} | 0.85 (0.60, 1.20) | 1.12 (0.77, 1.63) | 0.96 (0.59, 1.56) | 1.59 (0.92, 2.74) |
| 4th (highest) | 0.65 (0.45, 0.93)⁵ | 0.84 (0.56, 1.25) | 0.52 (0.30, 0.90)⁵ | 0.91 (0.49, 1.67) |
| p for trend⁶ | 0.01 | 0.4 | 0.03 | 0.9 |
| Folic acid | | | | |
| 1st (lowest) | 1.00 | 1.00 | 1.00 | 1.00 |
| 2^{nd} | 0.94 (0.67, 1.32) | 1.13 (0.78, 1.64) | 0.77 (0.48, 1.24) | 0.93 (0.55, 1.58) |
| 3^{rd} | 0.63 (0.44, 0.89)⁵ | 0.79 (0.54, 1.16) | 0.58 (0.35, 0.95)⁵ | 0.75 (0.43, 1.28) |
| 4th (highest) | 0.64 (0.45, 0.91)⁵ | 0.89 (0.60, 1.31) | 0.46 (0.27, 0.78)⁵ | 0.68(0.38, 1.22) |
| p for trend⁶ | 0.002 | 0.2 | 0.002 | 0.1 |
| α Carotene | | | | |
| 1st (lowest) | 1.00 | 1.00 | 1.00 | 1.00 |
| 2nd | 0.77 (0.54, 1.09) | 0.87 (0.60, 1.26) | 0.98 (0.60, 1.60) | 1.22 (0.71, 2.09) |
| 3rd | 0.82 (0.58, 1.16) | 1.00 (0.69, 1.45) | 0.74 (0.43, 1.25) | 1.01 (0.57, 1.80) |
| 4th (highest) | 0.75 (0.53, 1.06) | 0.92 (0.63, 1.34) | 0.88 (0.53, 1.45) | 1.26 (0.72, 2.21) |
| p for trend⁶ | 0.2 | 0.8 | 0.4 | 0.6 |
| β Carotene | | | | |
| 1st (lowest) | 1.00 | 1.00 | 1.00 | 1.00 |
| 2nd | 1.01 (0.71, 1.41) | 1.28 (0.88, 1.88) | 0.85 (0.52, 1.37) | 1.24 (0.72, 2.13) |
| 3rd | 0.85 (0.60, 1.20) | 1.21 (0.82, 1.77) | 0.76 (0.46, 1.24) | 1.23 (0.72, 2.13) |
| 4th (highest) | 0.71 (0.50, 1.02) | 0.98 (0.66, 1.47) | 0.58 (0.34, 0.97)⁵ | 1.01 (0.56, 1.81) |
| p for trend⁶ | 0.04 | 0.9 | 0.04 | 0.9 |
| β Cryptoxanthin | | | | |
| 1st (lowest) | 1.00 | 1.00 | 1.00 | 1.00 |
| 2nd | 0.80 (0.57, 1.13) | 0.82 (0.57, 1.18) | 1.36 (0.82, 2.24) | 1.33 (0.78, 2.28) |
| 3rd | 0.72 (0.51, 1.02) | 0.81 (0.56, 1.18) | 0.95 (0.56, 1.63) | 0.97 (0.55, 1.72) |
| 4th (highest) | 0.92 (0.66, 1.30) | 0.97 (0.67, 1.41) | 1.05 (0.62, 1.78) | 1.28 (0.73, 2.26) |
| p for trend⁶ | 0.5 | 0.9 | 0.7 | 0.7 |
| Lycopene | | | | |
| 1st (lowest) | 1.00 | 1.00 | 1.00 | 1.00 |
| 2nd | 1.27 (0.90, 1.80) | 1.50 (1.02, 2.19) | 0.88 (0.53, 1.46) | 1.06 (0.62, 1.83) |
| 3rd | 1.26 (0.90, 1.79) | 1.67 (1.14, 2.45) | 1.01 (0.62, 1.64) | 1.40 (0.82, 2.40) |
| 4th (highest) | 0.83 (0.58, 1.19) | 0.96 (0.64, 1.42) | 0.70 (0.42, 1.17) | 0.75 (0.42, 1.33) |
| p for trend⁶ | 0.3 | 0.9 | 0.3 | 0.6 |
| Copper | | | | |
| 1st (lowest) | 1.00 | 1.00 | 1.00 | 1.00 |
| 2nd | 0.78 (0.55, 1.10) | 0.93 (0.64, 1.35) | 0.63 (0.38, 1.03) | 0.75 (0.43, 1.30) |
| 3rd | 0.71 (0.50, 1.01) | 0.91 (0.62, 1.34) | 0.67 (0.41, 1.09) | 1.02 (0.60, 1.75) |
| 4th (highest) | 0.69 (0.49, 0.99)⁵ | 0.90 (0.61, 1.33) | 0.57 (0.34, 0.95)⁵ | 0.90 (0.51, 1.59) |
| p for trend⁶ | 0.03 | 0.6 | 0.04 | 1.0 |

¹Calorie adjusted nutrients, including supplements;
²123 children who were reported to wheeze on either the first or the second year only were excluded from the recurrent wheeze analyses;
³adjusted for sex, maternal age, maternal asthma, paternal asthma, family income, passive smoke exposure, breastfeeding, and other children <12 yr in the home;
⁴adjusted for bw, sex, maternal age, maternal pre-pregnancy BMI, maternal asthma, paternal asthma, family income, passive smoke exposure, breastfeeding, and other children <12 yr in the home;
⁵p < 0.05 for the comparison with the lowest quartile of intake;
⁶for trend obtained from trend test (Wald) in logistic regression models

Maternal intakes of lutein+zeaxanthin in the highest quartile compared with the lowest quartile were inversely associated with the presence of respiratory infections in the 2-year old children (OR=0.56, 95% CI=0.37, 0.85; p=0.01 for trend across quartiles) in a multivariable model adjusting for potential confounders. Total maternal intakes of all the other antioxidants, in particular, vitamin C, vitamin E, and zinc, were not associated with respiratory infections in the children.

Because antioxidants come from both foods and supplements (mostly in the form of multivitamin preparations), we examined the effects of vitamin E and zinc from foods and from supplements separately (Table 7). Vitamin E intakes from both foods and supplements were inversely associated with both wheeze outcomes, and the contribution of supplements appeared to be more strongly associated with the outcomes. Zinc intakes from both foods only and supplements only were inversely associated with any wheeze with similar effect estimates. Higher zinc intake from supplements only was also inversely associated with recurrent wheeze. The separate effects of antioxidants from foods only and from supplements only did not remain statistically significant in multivariable models.

In summary, we have found that higher maternal intakes of antioxidant nutrients during pregnancy, in particular vitamin E and zinc, are associated with lower risks for wheezing illnesses in 2-year old children. These effects were independent of a number of potential confounders, including maternal asthma status, breastfeeding, and infant multivitamin intake in the first year. Antioxidant nutrients from both foods and supplements contributed to these effects.

TABLE 7
Associations of nutrients from foods only and from supplements
only with wheezing outcomes at 2 years of age¹

| | Any Wheeze | | | | Recurrent Wheeze | | | |
|---|---|---|---|---|---|---|---|---|
| Nutrients | n = 1,161 | | | | n = 1,038 | | | |
| (Quartiles of | Foods Only | | Supplements Only | | Foods Only | | Supplements Only | |
| intake) | OR (95% CI) | | OR (95% CI) | | OR (95% CI) | | OR (95% CI) | |
| Vitamin E | | | | | | | | |
| 1st (lowest) | 1.00 | | 1.00 | | 1.00 | | 1.00 | |
| 2nd | 0.75 | (0.53, 1.06) | 0.86 | (0.61, 1.21) | 0.66 | (0.40, 1.07) | 0.83 | (0.52, 1.33) |
| 3rd | 0.67 | (0.47, 0.95)² | 0.70 | (0.50, 0.99)² | 0.58 | (0.35, 0.95)² | 0.50 | (0.30, 0.84)² |
| 4th (highest) | 0.69 | (0.49, 0.98)² | 0.63 | (0.45, 0.90)² | 0.61 | (0.37, 1.01) | 0.56 | (0.34, 0.93)² |
| p for trend³ | 0.03 | | 0.006 | | 0.04 | | 0.006 | |
| Zinc | | | | | | | | |
| 1st (lowest) | 1.00 | | 1.00 | | 1.00 | | 1.00 | |
| 2nd | 0.85 | (0.60, 1.19) | 0.98 | (0.70, 1.37) | 0.70 | (0.43, 1.17) | 0.94 | (0.58, 1.50) |
| 3rd | 0.60 | (0.42, 0.85)² | 0.67 | (0.47, 0.95)² | 0.65 | (0.40, 1.07) | 0.59 | (0.35, 0.98)² |
| 4th (highest) | 0.60 | (0.42, 0.85)² | 0.63 | (0.45, 0.90)² | 0.68 | (0.42, 1.12) | 0.51 | (0.30, 0.87)² |

| | | | | |
|---|---|---|---|---|
| p for trend³ | 0.0008 | 0.002 | 0.1 | 0.004 |

¹ORs and 95% CI from univariate logisitics regression models
²p < 0.05 for the comparsion with the lowest quartile of intake from logistic regression models
³p for trend obtained from trend test (Wald) in logistic regression models

### Low Serum Vitamin D Levels are Associated with Increased Asthma Exacerbations Among Children Using Regular Inhaled Corticosteroids

Among steroid-resistant asthmatics, vitamin D supplementation was found to improve the cellular response to dexamethasone. 25-Hydroxyvitamin D (25(OH)D) is an index of vitamin D status that reflects dietary intake and sun exposure, and levels between 30 and 40 ng/ml are considered sufficient for overall health. We sought to determine whether levels of 25(OH)D affect the response to inhaled corticosteroids among childhood asthmatics.

We measured 25(OH)D levels in stored serum (collected on entry into the trial) from 305 childhood asthmatics who were randomized to the inhaled corticosteroid arm of the multi-center Childhood Asthma Management Program. We then analyzed baseline 25(OH)D levels for their association with severe asthma exacerbations (defined as emergency room visits or hospitalizations for asthma) over the course of the 4-year trial.

Mean 25(OH)D level for the 305 children was 40.7 (±15.1) ng/ml, range 22.4-75.1 ng/ml. African American race, higher BMI, more northern latitude, and collection of specimens in the winter or spring were all strong and significant predictors of lower 25(OH)D levels. In multivariable logistic regression models adjusting for age, sex, height, and baseline FEV₁, children with 25(OH)D levels ≦30 ng/ml were more likely to have severe asthma exacerbations over the course of the 4-year trial (OR=1.76, 95% CI=1.01, 3.09). There were no associations between 25(OH)D and lung function, bronchodilator response, or airway responsiveness.

Insufficient levels of 25(OH)D are associated with increased risk for severe exacerbations of asthma among children on inhaled corticosteroids. Whether supplementation with vitamin D can decrease this risk needs further study.

### OTHER EMBODIMENTS

All publications and patents cited in this specification are herein incorporated by reference as if each individual publication or patent were specifically and individually indicated to be incorporated by reference. Use of singular forms herein, such as “a” and “the,” does not exclude indication of the corresponding plural form, unless the context indicates to the contrary. Although the invention has been described in some detail by way of illustration and example for purposes of clarity of understanding, it will be readily apparent to those of ordinary skill in the art in light of the teachings of the invention that certain changes and modifications may be made thereto without departing from the spirit or scope of the appended claims.

Other embodiments are within the following claims.

What is claimed is:

1. A method of treating or preventing a disease or condition of the immune system or an infectious disease, comprising:
(i) administration of at least 400 IU of vitamin D; optionally in combination with
(ii) administration of one or more antioxidants.

2. (canceled)

3. The method of claim 1, wherein
400 IU to 10,000 IU of vitamin D is administered;
1,000 IU to 6,000 IU of vitamin D is administered; or
2,000 IU to 4,000 IU of vitamin D is administered.

4-5. (canceled)

6. The method of claim 1, wherein the one or more antioxidants is vitamin E.

7. The method of claim 6, wherein
200 IU to 4;000 IU of vitamin E is administered;
300 IU to 2,500 IU of vitamin E is administered;
400 IU to 1,000 IU of vitamin E is administered; or
500 IU to 800 IU of vitamin E is administered.

8-10. (canceled)

11. The method of claim 1, wherein the one or more antioxidants is zinc.

12. The method of claim 1, wherein
the vitamin D and/or one or more antioxidants is administered daily;
the vitamin D and/or one or more antioxidants is administered in a single dosage form;
the vitamin D and/or one or more antioxidants are administered together in the form of a multivitamin;
the vitamin D is administered orally; and/or
the vitamin D is administered by inhalation.

13-14. (canceled)

15. The method of claim 1, wherein the disease or condition is selected from the group consisting of asthma, an autoimmune disease, allergic rhinitis, eczema, psoriasis, food allergy, type-1 diabetes mellitus, ulcerative colitis, Crohn's disease, rheumatoid arthritis, multiple sclerosis, systemic lupus erythematosus, bullous pemphigoid, myasthenia gravis, tuberculosis, and a parasitic infection.

16. The method of claim 1, wherein the vitamin D and/or one or more antioxidants is administered to (i) a pregnant woman and the treatment or prevention of a disease or condition of the immune system is directed to the child to whom the pregnant woman gives birth, or (ii) a child having steroid-resistant asthma.

17-22. (canceled)

23. A kit or pharmaceutical container comprising at least 400 IU of vitamin D in a single dosage form and/or at least 200 IU of vitamin E in single dosage form.

24. The kit or pharmaceutical container of claim 23, comprising multiple single dosages of vitamin D.

25. The kit or pharmaceutical container of claim 23, wherein
the single dosage of vitamin D comprises 400 IU to 10,000 IU of vitamin D;
the single dosage of vitamin D comprises 1,000 IU to 6,000 IU of vitamin D; or
the single dosage of vitamin D comprises 2,000 IU to 4,000 IU of vitamin D.

26-27. (canceled)

28. The kit or pharmaceutical container of claim 23, further comprising said at least 400 IU of vitamin D in single dosage form and one or more antioxidants.

29. The kit or pharmaceutical container of claim 28, wherein the one or more antioxidants is vitamin E and/or zinc.

30. (canceled)

31. The kit or pharmaceutical container of claim 29, comprising multiple single dosages of vitamin E.

32. The kit or pharmaceutical container of claim 23, wherein
the single dosage of vitamin E comprises 200 IU to 4,000 IU of vitamin E;
the single dosage of vitamin E comprises 300 IU to 2,500 IU of vitamin E;
the single dosage of vitamin E comprises 400 IU to 1,000 IU of vitamin E; or
the single dosage of vitamin E comprises 500 IU to 800 IU of vitamin E.

33-35. (canceled)

36. A device comprising vitamin D for administration by inhalation.

37. The device of claim 36, wherein the device is a nebulizer, spray device, or other inhaler.

38-46. (canceled)
